(19) United States
(12) Patent Application Publication
(10) Pub. No.: US 2025/0058048 A1
(43) Pub. Date: Feb. 20, 2025

(54) AUTOINJECTOR WITH PRODUCT CONTAINER RECEPTACLE

(71) Applicant: Ypsomed AG, Burgdorf (CH)

(72) Inventors: Gabriel Kalbermatter, Burgdorf (CH); Mario Bernhard, Burgdorf (CH); Christian Schrul, Oberburg (CH); Nicolas Gruenig, Bern (CH); Marcel Allenspach, Burgdorf (CH); Markus Tschirren, Burgdorf (CH)

(21) Appl. No.: 18/936,015

(22) Filed: Nov. 4, 2024

Related U.S. Application Data
(63) Continuation of application No. PCT/EP2023/061593, filed on May 3, 2023.

(30) Foreign Application Priority Data
May 12, 2022 (EP) 22173096.3

(51) Int. Cl. A61M 5/20; A61M 5/24; A61M 5/315

(52) U.S. Cl. CPC A61M 5/2033; A61M 5/31583; A61M 2005/2403

(57) ABSTRACT

A product container receptacle and an autoinjector may include: a syringe holder for accommodating a pre-filled, ready-to-use syringe defining a longitudinal axis, with a cylindrical product container that narrows at its distal end via a shoulder, and a needle protection cap. The syringe holder includes a distally, coaxially arranged guide sleeve on the inner wall of which a blocking surface is formed; an insert having a snap arm, which is radially deflectable with respect to the longitudinal axis from a relaxed position and has a snap hook; and a detachable device cap in which the insert is connectably accommodated. The insert is movable relative to the device cap from a first latching position to a second latching position, and the connection of the device cap to the insert, in at least the second latching position, is configured to latch and form-fit with respect to tensile forces.

### CROSS REFERENCE TO RELATED APPLICATIONS

This application claims priority to International Patent Application No. PCT/EP2023/061593, filed May 3, 2023, entitled “AUTOINJECTOR WITH PRODUCT CONTAINER RECEPTACLE,” which in turn claims priority to European Patent Application No. 22173096.3, filed May 12, 2022, entitled “AUTOINJECTOR WITH PRODUCT CONTAINER RECEPTACLE”, each of which is incorporated by reference herein, in the entirety and for all purposes.

### TECHNICAL FIELD

The present invention relates to the field of medical injection devices for administering liquid substances, in particular medicaments or medical substances, such as insulin and hormone preparations. The invention relates to an autoinjector with an energy store for ejecting a predetermined dose from a product container that is used one time.

### BACKGROUND

Injection devices or injection apparatuses for the simplified administration of a substance comprise, inter alia, so-called autoinjectors which have an energy store with which the discharge process can be carried out automatically, i.e., without a force to be supplied or exerted externally by a user. The energy store advantageously stores the energy required for an automatic substance delivery in mechanical form. Such an energy store can be a spring which is installed in a tensioned state in the injection device and delivers energy by expansion. The energy is delivered to a piston rod or a pressure element, which pushes a piston into a product container. The energy store may also be provided in order to automate the process of inserting an injection needle. Alternatively, the insertion process can take place manually, i.e., exclusively by a user, without using energy stored in the injection device for this purpose.

The injection device may comprise a product container holder for accommodating a product container, wherein the product container can be held in the product container holder radially, axially, and preferably also in a rotationally fixed manner. The product container holder may be connected to the housing of the injection apparatus in an axially and rotationally fixed manner or may be movable relative to the housing during an insertion and/or needle retraction process. The product container may be a cartridge for the repeatedly detachable connection to disposable injection needles or a disposable ready-to-use syringe with an injection needle non-detachably connected thereto. The product container has a hollow cylindrical product container portion which displaceably mounts a piston or plunger. The piston can form a sealing gap with the inner circumference of the product container portion and can be displaced in a distal direction by means of a piston rod in order to dispense product from the product container via the injection needle.

The injection device may have a needle protection sleeve which, after injection has taken place, projects distally beyond the distal end of the injection needle or is displaced relative to the housing into this position while expanding a needle protection sleeve spring, in order to prevent accidental access to the injection needle and to reduce the risk of injury. In an autoinjector, the needle protection sleeve can also serve as a trigger element for triggering the product ejection process, wherein the needle protection sleeve is displaced relative to the housing in the proximal direction for this purpose. Alternatively, the triggering of the autoinjector can be achieved by actuating a trigger button of the autoinjector, wherein the needle protection sleeve serves at least as a visual protection before the autoinjector is used.

Patent application WO 2016/205963 describes an exemplary autoinjector comprising a housing with a longitudinal axis and a product container arranged axially fixedly in the housing. A spiral spring or mainspring in which energy for the automatic discharge of product can be stored is connected via a first end to the housing, and via a second end is connected in a rotationally fixed manner to a drive element in the form of a rotating threaded rod arranged coaxially with the longitudinal axis. The threaded rod engages via a thread in a propulsion member in the form of a propulsion sleeve, which propulsion member is not rotating in the housing and which propulsion sleeve moves the plunger of the product container at an approximately constant discharge rate in the distal direction during a displacement.

A mainspring as a drive is characterized by high forces and is thus suitable for a delay-free start of discharge, even with autoinjectors with a long storage period. A variable thread pitch of the threaded rod can compensate for a variable characteristic curve of the mainspring in order to ensure the most constant possible discharge force. This means that even large discharge volumes of 5 mL or more can be discharged evenly and continuously within a maximum of 60 seconds. With an extended discharge time of more than 10 s compared to conventional autoinjectors, the user should be able to ensure that the injection is proceeding as intended at all times in order to avoid inadvertently stopping the injection prematurely.

Patent application WO 2012/173554 shows a rotating display at the proximal end of an autoinjector, which is driven by a torsion spring during discharge. The display comprises a plurality of segments with different colors for signaling an initial state prior to the commencement of discharge and a final state after discharge has taken place. The segments are visible through at least one window at the proximal end of the autoinjector, the extension of which corresponds to the angular range of a segment.

The patent application published as CH714527A2 discloses an injection device with a cap for removing a needle protection cap from a product container and a method for assembling an injection device, the cap comprising an engagement element to cause removal of the needle protection cap from the product container when the cap is removed from the injection device.

### SUMMARY

It is an object of the present disclosure to create a display for an autoinjector of the type mentioned above, which can visually display to the user the persistence or progression of a discharge in a wide variety of grip positions. The display may be designed in such a way that even in unfavorable-in particular, unintended-grip positions of a user's hand on the autoinjector, the user can recognize the persistence of the discharge without having to change the grip position. Another object is to create an improved and more cost-effective autoinjector. The assembly of the autoinjector or its units and the insertion of a product container during final assembly may be more reliable, and the operation and handling of the autoinjector may be simpler and safer. The pre-assembly of units, which is separate in time and place from the final assembly of the autoinjector, as well as the storage, transport, and supply of the same, may be simpler and safer; in particular, the integrity of the units may be maintained during transport and supply.

The object is achieved by devices having the features of the present disclosure.

An autoinjector according to the present disclosure comprises a one-piece or multi-part housing with a longitudinal axis and a pre-filled ready-to-use syringe with a product container and an injection needle or cannula which is non-detachably fastened thereto. The ready-to-use syringe is axially non-displaceably accommodated in the housing, wherein a tip of the injection needle projects by at least one piercing depth in the distal direction beyond a distal housing end. The autoinjector furthermore comprises a torsion spring pretensioned for the single discharge of a maximum content of the product container, a drive element, a propulsion element, and a needle protection sleeve. For discharging liquid from the product container through the injection needle, the torsion spring puts the drive element into rotation about the longitudinal axis, and the rotating drive element causes a linear movement of the propulsion element in order to displace a piston in the product container. When the autoinjector is pressed against a point of injection, and the injection needle is caused to pierce the point of injection as a result, the needle protection sleeve is moved in the proximal direction by an actuation stroke, thereby starting or allowing a discharge of liquid. The actuation stroke of the needle protection sleeve corresponds here to at least the piercing depth of the injection needle.

The autoinjector finally comprises a display for signaling the persistence of a discharge, having a display element, which is driven by the drive element, rotates about the longitudinal axis, and has an optical contrast pattern, and a window at the proximal end of the autoinjector, through which window the rotating contrast pattern is visible. The window is completely circumferential by 360° about the longitudinal axis, i.e., not interrupted by a frame or web parallel to the longitudinal axis, which creates a dead angle for the view on the display element. The window is made of a transparent and preferably stable or load-bearing material. As long as even a small part of the window remains visible, i.e., even if the user clumsily covers a large part of the window with the hand holding the autoinjector, e.g., by supporting the proximal end with the heel of their hand, the rotating display element can be recognized.

The contrast pattern is at most discretely rotationally symmetrical about the longitudinal axis, but not continuously rotationally symmetrical, so that a rotation of the contrast pattern can also be recognized. The contrast pattern comprises at least one area or one graphic element in a color or gray tone different from a background color, preferably in the form of graphic elements repeating in the direction of rotation, such as parallel lines.

An autoinjector according to the present disclosure is suitable for discharging the amount of product contained in the product container during a discharge time of more than 10 s—preferably more than 20 s or 30 s—which is longer than that of known autoinjectors. As a result, even with product quantities of more than 2.25 mL and preferably at least 3 mL or 4 mL, a continuous or average discharge rate is relatively low, so that the injected quantity of medicament can be continuously absorbed or resorbed by the subcutaneous tissue. Therefore, maintaining a minimum holding time between the end of the discharge and moving the autoinjector away from the puncture site is less critical. Accordingly, no visual, acoustic, or tactile signaling is required to display the end of a holding time to the user.

In a preferred embodiment, the rotating display element is driven directly or immediately—in particular, without gearing—by the drive element. The display element thus rotates at the same rotational speed as the drive element.

In a further preferred embodiment, the display element carries out less than one revolution—preferably less than half a revolution or even less than a third of a revolution—per second during discharge. This ensures that the rotational movement of the contrast pattern can be easily followed by the eye and does not, for instance, blur into an indeterminate, rotationally symmetrical pattern for the viewer.

In a preferred embodiment, neither on the window nor on the adjoining housing is an optical marker for determining a relative rotational position of the contrast pattern provided. The display element does not display a start or end status, nor does it provide any relative information on the progress of the discharge; it is used solely to signal the progress or persistence of the discharge.

In a preferred embodiment, the rotating display element and the housing or the window are designed to create a discharging sound, thereby additionally signaling to the user the persistence of the discharge process acoustically. Preferably, the discharging sound is a continuous clicking sound, which is created by a grid on the window or on the housing and a radially or axially flexible engagement element engaging therein on the display element, or, conversely, by a rotating grid on the display element and a stationary engagement element. In this case, as with a purely optical movement indicator, a separate optical, acoustic, and/or tactile signaling of the discharge, e.g., by a mechanically or electronically generated signal, can be omitted.

In a preferred embodiment, the window has a first, cylindrical region parallel to the longitudinal axis, and a second region, seamlessly adjoining the first in the proximal direction, with a constantly or continuously decreasing diameter. The first region enables a view of the display element from any lateral viewing direction perpendicular to the longitudinal axis, and the second region enables a view of the display element in the distal viewing direction from behind, or from above when the autoinjector is held vertically. The second region is rounded or constricted in a proximal direction and can end in a third region perpendicular to the longitudinal axis. The display element can have a convex display surface following the course of the window or a cone-shaped display surface with a medium inclination with respect to the longitudinal axis. Preferably, the window is rotationally symmetrical with respect to the longitudinal axis, i.e., with circular cross-sections in sectional planes perpendicular to the longitudinal axis.

In an advantageous variant, the autoinjector comprises a needle protection sleeve, which projects distally over the housing in the delivery state of the autoinjector and is pretensioned in the distal direction by a needle protection spring. When the autoinjector is pressed against a point of injection, the needle protection sleeve carries out an actuation movement in the proximal direction and, when the autoinjector is removed from the point of injection, carries out a needle protection movement in the distal direction, in order to laterally surround and radially cover the injection needle with a sleeve-shaped, preferably rotationally symmetrical, portion. The needle protection sleeve has, at a distal end, an annular flange or base as an enlarged contact surface for the tissue around the point of injection. The flange is permanently connected to the sleeve-shaped portion and is preferably formed in one piece with it. A maximum diameter of the flange is greater than a maximum diameter of the sleeve-shaped portion.

Preferably, the outer edge or the periphery of the flange is adapted to the shape of a distal opening in the housing. In the inserted state, the flange thus forms a closure for the housing. The flange is also preferably designed to be concavely curved, so that the outer edge is further distal than the transition between the flange and the sleeve-shaped portion. Compared to a flange with a strictly flat, ring-shaped contact surface with an identical outer diameter, this, firstly, makes it less likely that the attached autoinjector will tilt, and, secondly, the concentration of the pressure load on the periphery of the flange furthest away from the puncture site results in less counter-pressure in the tissue and therefore less pain for the user.

Further preferably, the autoinjector comprises a threaded rod as the drive element and a propulsion sleeve with an internal thread as the propulsion element-alternatively, a drive sleeve with an internal thread as the drive element and a threaded rod as the propulsion element-the propulsion element having a groove or a cam as an axial guide element for an exclusively linear propulsion movement in the housing. The autoinjector is preferably dimensioned for accommodating a pre-filled ready-to-use syringe comprising the product container and the injection needle and has a filling volume of at least 3 mL—preferably at least 5 mL.

In summary and in other words, an autoinjector according to aspects of the invention can be designed as follows:
Autoinjector comprising
   a housing (10a, 10b) with a longitudinal axis L and for accommodating a product container (11),
   a propulsion member (22) and a rotating drive element (21) for moving the propulsion member (22) in the longitudinal direction and for automatically discharging a liquid product contained in the product container (11) through an injection needle (11b),
   a display for signaling the persistence of a discharge, comprising a rotating display element (25a) driven by the drive element (21) and having an optical contrast pattern () and comprising a window (25b) at the proximal end of the autoinjector, through which window the rotating contrast pattern is visible, characterized in that the window (25b) is a window completely circumferential about the longitudinal axis.

Autoinjector in which the rotating display element (25a) is driven directly by the drive element (21).

Autoinjector in which the display element (25a) carries out less than one, preferably less than half of one, revolution per second during a discharge.

Autoinjector in which no optical marking for determining a rotational position of the contrast pattern is provided.

Autoinjector in which the rotating display element (25a) is designed to create a discharging sound.

Autoinjector in which the window (25b) has a first, cylindrical region parallel to the longitudinal axis, and a second region, seamlessly adjoining the first in the proximal direction, with a constantly decreasing diameter.

Autoinjector, further comprising
a needle protection sleeve (14), which projects distally over the housing in the delivery state of the autoinjector, wherein, when the autoinjector is pressed against a point of injection, the needle protection sleeve (14) carries out an actuation movement in the proximal direction and, when the autoinjector is removed from the point of injection, carries out a needle protection movement in the distal direction in order to laterally surround the injection needle (11b) with a sleeve-shaped portion (14b),
   the needle protection sleeve (14) having an annular flange (14c) at a distal end for contact with the point of injection, with a maximum diameter that is greater than the maximum diameter of the sleeve-shaped portion (14b).

Autoinjector in which an outer edge of the flange (14c) is adapted to a distal opening in the housing (10b).

Autoinjector in which the flange (14c) is concavely curved.

Autoinjector in which the drive element (21) is a threaded rod, and the propulsion element (22) is a propulsion sleeve with an axial guide element for an exclusively linear propulsion movement in the housing (10b).

Autoinjector further having a pre-filled ready-to-use syringe comprising the product container (11) and the injection needle (11b), and having a filling volume of at least 3 mL, preferably at least 5 mL.

Further preferred embodiments of the present disclosure, in particular of product container receptacles for an improved autoinjector, are described below.

In a preferred embodiment, a product container receptacle for an autoinjector comprises a syringe holder designed to accommodate a pre-filled ready-to-use syringe defining a longitudinal axis which extends from distal to proximal, with a cylindrical product container which narrows at its distal end via a shoulder, and with a needle protection cap, the syringe holder having a distally coaxially arranged guide sleeve, on the inner wall of which a blocking surface is formed; in particular, the blocking surface can also be formed by individual or connected ribs or spokes. Furthermore, a sleeve-shaped insert having a snap arm which is radially deflectable with respect to the longitudinal axis from a relaxed position and has a snap hook, and a device cap in which the insert is connectably accommodated, wherein the insert is movable from a first latching position to a second latching position relative to the device cap, and the connection of the device cap to the insert, in at least the second latching position, is designed to be latching and form-fitting with respect to tensile forces along the longitudinal axis, wherein the snap arm with snap hook is radially deflectable in the first latching position and is prevented from radial deflection by the blocking surface in the second latching position.

Preferably, the product container receptacle is further developed in that the snap arm with snap hook can be deflected radially outwards by the needle protection cap when the insert is in the first latching position. This means that the joining forces when assembling the ready-to-use syringe are minimal, and the closure security of the product container is not compromised.

Preferably, the product container receptacle is further developed in that the snap arm with snap hook is designed to axially entrain the needle protection cap in the distal direction in a form-fitting manner when the insert is in the second latching position. This allows a safe separation of the needle protection cap when pulled off the syringe, and there are no unnecessarily high pulling-off forces resulting from additional friction.

Preferably, the product container receptacle is further developed in that the syringe holder is designed such that a distal portion of the ready-to-use syringe, in particular the needle protection cap, is axially movable in the guide sleeve, and this mobility of the ready-to-use syringe in the distal direction is limited form-fittingly. This means that the ready-to-use syringe is radially centered during assembly and can be positioned at the intended axial location in the device without play.

Preferably, the product container receptacle is further developed in that the guide sleeve with its proximal end forms a stop for the cylindrical product container, in particular for its shoulder. This ensures that the ready-to-use syringe is positioned at the intended axial location in the device during assembly.

Preferably, the product container receptacle further comprises a needle protection sleeve with a retaining catch, wherein, in the first latching position, the retaining catch connects the insert force-fittingly relative to the needle protection sleeve by engaging in the first snap connection, and/or, in the second latching position, the retaining catch connects the insert force-fittingly relative to the needle protection sleeve by engaging in the second snap connection or moving in the first snap connection. This keeps the needle protection sleeve or insert in the intended axial and rotational position in the autoinjector, and the retaining force also increases security against unintentional triggering due to high accelerations-for example, during transport or if the device falls to the ground.

Preferably, the product container receptacle is further developed in that the device cap can be separated from the product container receptacle by a pulling-off movement, wherein the insert is entrained, and the force-fitting connection between the needle protection sleeve and the insert is released. This results in a defined initial resistance, which reduces unintentional release or unwanted play.

Preferably, the product container receptacle further comprises a housing or housing part, wherein the syringe holder is accommodated therein immovably, at least with respect to axial tension in the distal direction or axial pressure from the proximal direction or is formed integrally therewith. This ensures that the syringe holder is held securely in the intended axial location in the device when the needle protection cap is pulled off or during discharge.

Preferably, in a further embodiment of the invention, the product container receptacle is formed in that the insert is axially movable from proximal to distal relative to the device cap, from the first latching position to the second latching position, and is guided in a rotationally fixed manner by a rib in a guide groove, and/or the snap cam changes from the first snap connection to the second snap connection. As a result, the insert is captively secured in a pre-assembly position in a subunit of the device before assembly of the ready-to-use syringe and can be secured in its final assembly position during assembly of the ready-to-use syringe. The movement of the insert can be caused by a tool or by another part that moves axially during assembly. In particular, the movement from the first latching position to the second latching position can be caused by or derived from the joining movement of the ready-to-use syringe.

Preferably, the product container receptacle is further developed in that the insert can be pushed axially in the distal direction by the shoulder via a proximally provided end face. As a result, the insert is brought into its final assembly position directly by the axial joining movement of the ready-to-use syringe during assembly, eliminating the need for a complex tool that would have to reach into the interior of the device.

Preferably, the product container receptacle is further developed in that the blocking surface formed on the inner wall of the guide sleeve is limited to an axial portion. This portion is selected such that, at the beginning of the joining movement of the ready-to-use syringe during assembly, the snap arm with snap hooks can be radially deflected freely and with little friction by the outer profile of the needle protection cap until it elastically snaps in behind the proximal edge of the needle protection cap, and, towards the end or after the joining movement, remains form-fittingly secured in this position against radial breakout by the blocking surface or another suitable blocking geometry.

Preferably, in a further embodiment of the invention, the product container receptacle is formed in that the insert is movable from the first latching position to the second latching position relative to the device cap in a rotational manner about the longitudinal axis and is axially fixedly guided by a snap cam in a snap groove and/or can be secured in a rotationally fixed manner by the groove in the second latching position. As a result, the insert is captively secured in a pre-assembly position in a subunit of the device before assembly of the ready-to-use syringe and can be secured in its final assembly position during assembly of the ready-to-use syringe.

Preferably, the product container receptacle is further developed in that the insert is movable rotationally about the longitudinal axis via one or more distally provided assembly cams. A torque can be temporarily input via the assembly cams by means of a turning tool or by means of static structures in the assembly device (relative to which the device can be rotated), which causes the rotational movement. For this purpose, openings are provided in the device cap.

Preferably, the product container receptacle is further developed in that the blocking surface formed on the inner wall of the guide sleeve has one or more recesses in sectors. This recess is selected such that, during the joining movement of the ready-to-use syringe during assembly, the snap arm with snap hook can be radially deflected freely by the outer profile of the needle protection cap until it elastically snaps in behind the proximal edge of the needle protection cap, and, after the joining movement, the rotational movement of the insert is carried out as described above, so that the snap arm with snap hooks remains form-fittingly secured in this position against radial breakout by the blocking surface or another suitable blocking geometry.

Preferably, an autoinjector further developed according to the invention for the administration of a liquid product comprises
a product container receptacle according to the invention
a propulsion member and a drive element for moving the propulsion member in the longitudinal direction and for automatically discharging a liquid product contained in the product container of the pre-filled ready-to-use syringe through an injection needle, wherein the needle protection sleeve, which projects distally over the housing in the delivery state of the autoinjector, when the autoinjector is pressed against a point of injection, executes an actuating movement in the proximal direction and, when the autoinjector is removed from the point of injection, executes a needle protection movement in the distal direction in order to laterally surround the injection needle with a sleeve-shaped portion.

### BRIEF DESCRIPTION OF THE DRAWINGS

Embodiments of the present disclosure are described below in connection with the appended figures. These embodiments are intended to show basic possibilities and are in no way to be interpreted as limiting. In the drawings:

FIG. 1 illustrates an exploded view of the components of an autoinjector, according to the present disclosure,

FIGS. 2a and 2b illustrate two longitudinal cross-sections through the autoinjector of FIG. 1 before injection,

FIGS. 3a and 3b illustrate two isometric views of the display element of the autoinjector of FIG. 1,

FIG. 4 shows a view of the distal end of the needle protection sleeve of the autoinjector of FIG. 1,

FIG. 5 shows an exploded view of the components of a first embodiment of a product container receptacle, according to the present disclosure,

FIGS. 6a and 6d illustrate views of the product container receptable of FIG. 5 in the state before final assembly,

FIG. 6b1 illustrates a longitudinal cross-section along line Y-Y of FIG. 6d,

FIG. 6b2 illustrates a radial cross-section along line A-A of FIG. 6b1,

FIG. 6c1 illustrates a longitudinal cross-section along line X-X of FIG. 6a,

FIG. 6c2 illustrates a radial cross-section along line B-B of FIG. 6c1,

FIG. 7a illustrates the product container receptable of FIG. 5 in the state of joining the ready-to-use syringe,

FIG. 7b1 illustrates a longitudinal cross-section along line X-X of FIG. 7a,

FIG. 7b2 illustrates a radial cross-section along line A-A of FIG. 7b1,

FIGS. 8a and 8d illustrate views of the product container receptable of FIG. 5 in the state after the blocking movement of the insert,

FIG. 8b1 illustrates a longitudinal cross-section along line X-X of FIG. 8a,

FIG. 8b2 illustrates a radial cross-section along line A-A of FIG. 8b1,

FIG. 8c illustrates a longitudinal cross-section along line Y-Y of FIG. 8d,

FIG. 9a illustrates the product container receptable of FIG. 5 in the state before the needle protection cap is pulled off,

FIG. 9b illustrates a longitudinal cross-section along line X-X of FIG. 9a,

FIG. 9c1 illustrates a longitudinal cross-section along line Y-Y of FIG. 9d,

FIG. 9c2 illustrates a radial cross-section along line A-A of FIG. 9b,

FIG. 9d illustrates the product container receptable of FIG. 5 in the state after the needle protection cap is pulled off,

FIG. 10 shows an exploded view of the components of a second embodiment of a product container receptacle, according to the present disclosure,

FIGS. 11a and 11d illustrate the product container receptable of FIG. 10 in the state before final assembly,

FIG. 11b1 illustrates a longitudinal cross-section along line X-X of FIG. 11a,

FIG. 11b2 illustrates a proximal view of a distal end of the product container receptacle of FIG. 11a,

FIG. 11b3 illustrates a radial cross-section of the product container receptacle of FIG. 11a,

FIG. 11c1 illustrates a longitudinal cross-section along line Y-Y of FIG. 11d,

FIG. 11c2 illustrates a radial cross-section along line A-A of FIG. 11c1,

FIGS. 12a and 12c illustrate views of the product container receptable of FIG. 10 in the state of joining the ready-to-use syringe,

FIG. 12b1 illustrates a longitudinal cross-section along line A-A of FIG. 12a,

FIG. 12b2 illustrates a proximal view of a distal end of the product container receptacle of FIG. 12a,

FIG. 12b3 illustrates a radial cross-section of the product container receptacle of FIG. 12a,

FIGS. 13a and 13c illustrate the product container receptable of FIG. 10 in the state after the blocking movement of the insert,

FIG. 13b1 illustrates a longitudinal cross-section along line X-X of FIG. 13a,

FIG. 13b2 illustrates a proximal view of a distal end of the product container receptacle of FIG. 13a,

FIG. 13b3 illustrates a radial cross-section along line A-A of FIG. 13b1,

FIGS. 14a shows the device of FIG. 10 in the state after the needle protection cap is pulled off, and

FIG. 14b shows a longitudinal cross-section along line A-A of FIG. 14a.

### DETAILED DESCRIPTION

The term “product,” “medicament,” or “medical substance” in the present context includes any flowable medical formulation which is suitable for controlled administration by means of a cannula or hollow needle in subcutaneous or intramuscular tissue-for example, a liquid, a solution, a gel, or a fine suspension containing one or more medical active ingredients. A medicament can thus be a composition with a single active ingredient or a premixed or co-formulated composition with a plurality of active ingredients from a single container. The term includes in particular drugs, such as peptides (e.g., insulins, insulin-containing medicaments, GLP-1-containing preparations, as well as derived or analogous preparations), proteins and hormones, biologically obtained or active ingredients, active ingredients based upon hormones or genes, nutrient formulations, enzymes, and other substances both in solid (suspended) or liquid form. The term also includes polysaccharides, vaccines, DNA or RNA or oligonucleotides, antibodies or parts of antibodies, as well as suitable base substances, excipients, and carrier substances.

The term “distal” refers to a side or direction directed towards the front, piercing-side end of the administration apparatus or towards the tip of the injection needle. In contrast, the term “proximal” refers to a side or direction directed towards the rear end of the administration apparatus that is opposite the piercing-side end.

In the present description, the term “injector” is understood to mean an apparatus with which the injection needle is removed from the tissue after a controlled amount of the medical substance has been delivered. In contrast to an infusion system, the injection needle of an injector thus does not remain in the tissue for a longer period of several hours.

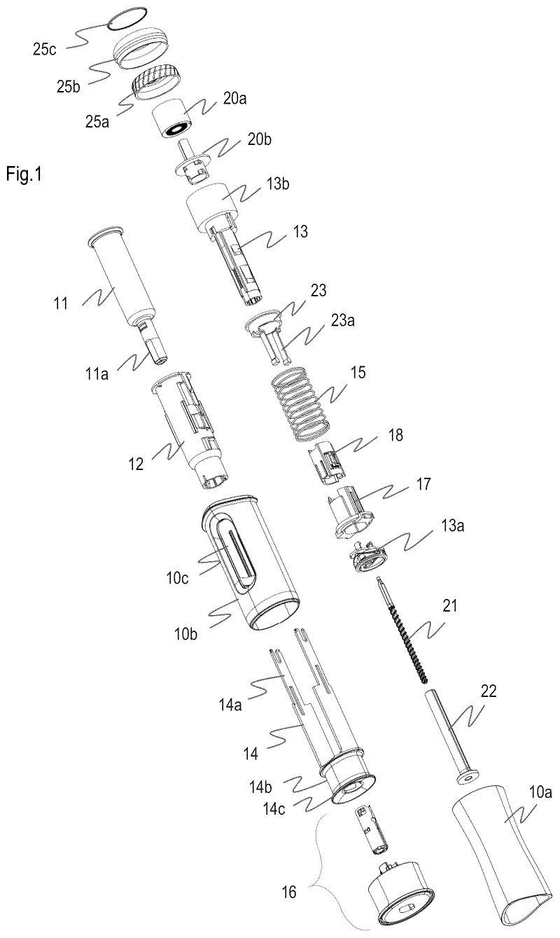

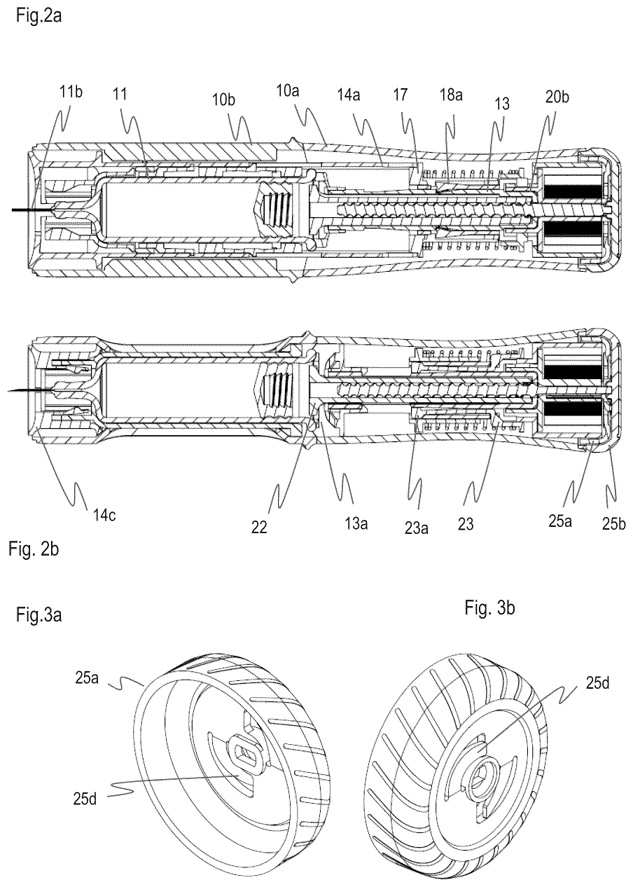

FIG. 1 is an exploded view of the components of an autoinjector according to the invention, and FIGS. 2a and 2b show longitudinal sections of the autoinjector according to FIG. 1 rotated by 90° to each other about the longitudinal axis in the inserted state and ready for discharge.

The autoinjector has a sleeve-shaped, elongated housing with a longitudinal axis L and comprising a distal housing part 10b and a proximal housing part 10a in the form of a handle that is non-detachably snap-fitted therewith. A product container in the form of a ready-to-use syringe 11 with an injection needle which is non-detachably fastened to the product container is held in a syringe holder 12, wherein the syringe holder is accommodated in the housing in an axially and rotationally fixed manner. The ready-to-use syringe 11 is pressed in a distal direction into engagement with a shoulder of the syringe holder 12 by a retaining spring portion 13a fixedly anchored in the housing part 10a. In relation to the housing part 10b, the ready-to-use syringe 11 is arranged such that the tip of the injection needle projects beyond the distal end of a proximal intermediate position of a needle protection sleeve 14 by a length corresponding to the subcutaneous or intramuscular piercing depth, and is at least laterally protected or covered by the needle protection sleeve 14 before and after the injection. When the injection needle is inserted into the point of injection along the longitudinal axis L, the needle protection sleeve 14 is pushed in the proximal direction by an actuation stroke and against the force of a needle protection spring 15, and thereby triggers a product discharge. For this purpose, the needle protection sleeve comprises two sleeve arms 14a, which are arranged offset or rotated by 90° about the longitudinal axis L with respect to two recesses 10c of the housing designated as viewing windows. After the injection has taken place, the needle protection sleeve 14 can be displaced relative to the housing part 10b from the actuated position along the longitudinal axis L in the distal direction to a needle protection position and can be blocked there against being pushed back again. The needle protection sleeve comprises a sleeve-shaped or hollow-cylindrical portion 14b and a flange 14c at the distal end. The cross-section of the hollow-cylindrical portion 14b is oval, as is the outer circumference of the flange, so that, in the present case, the flange has a constant width or radial extension.

A spring assembly comprises a spiral spring 20a and a spring coil 20b. The outer end of the spiral spring 20a is non-rotatably fastened to a spring sleeve 13b as part of a mechanism holder 13 fixedly anchored in the housing. The inner end of the spiral spring 20a is connected to the spring coil 20b in a rotationally fixed manner. The spring coil 20b comprises a spring shaft and a distal spring flange. The spiral spring 20a or the spring coil 20b puts a drive element 21 into a rotational movement and a propulsion element 22 into a preferably purely axial propulsion movement. For this purpose, a threaded element engages in a thread extending over the discharge stroke and having a variable thread pitch.

The ready-to-use syringe 11 comprises a cylindrical syringe body as a product container, at the distal end of which a hollow injection needle is fixedly connected to a syringe shoulder. The injection needle of the ready-to-use syringe is covered by a needle protection cap 11a, which is designed as a so-called rigid needle shield (RNS) and comprises a rubber-elastic needle protection element and a sheath made of hard plastic. The needle protection cap protects the injection needle against mechanical effects and contamination, and keeps the injection needle and the product sterile. At the distal end of the autoinjector, in the delivery state thereof, a two-part device cap or pull-off cap 16 is arranged, which is axially pulled off and/or twisted off and completely removed along with the needle protection cap 11a before the autoinjector is used.

A switching sleeve 17 is arranged in a positive-locking manner with a proximal end of the sleeve arms 14a of the needle protection sleeve 14 and with a distal end of the needle protection spring 15 and is at least partially surrounded by the latter. The switching sleeve 17 is preferably snap-fitted with the proximal end of the sleeve arms of the needle protection sleeve 14. The movement of the switching sleeve 17 in the distal direction is delimited by the retaining spring portion 13a, which in turn is snap-fitted together with the mechanism holder 13 after the switching sleeve 17 has been assembled. A locking sleeve 18 is arranged within and coaxially with the switching sleeve 17 and is coupled to the switching sleeve 17 via a saw tooth-shaped locking member 18a, which is resiliently attached to an arm pointing in the distal direction, in such a way that an actuation movement of the needle protection sleeve 14 and the switching sleeve 17 also moves the locking sleeve 18 proximally. By means of an additional proximal locking stroke of the locking sleeve 18 relative to the switching sleeve 17 into a proximal end position, the locking member 18a is reliably released by the switching sleeve 17 for movement inwards. Due to the spring effect of the arm, the locking member 18a engages behind a proximally directed edge of the autoinjector or latches into an axially fixed recess of the autoinjector and thus locks the locking sleeve 18 against a distal movement. When the autoinjector is removed from the puncture site, the switching sleeve 17 is pushed by the needle protection spring 15 in the distal direction over the locking member 18a, whereupon, as a result of the spring effect of the arm, the locking member engages behind a proximally directed edge of the switching sleeve 17 in a locking position and locks or blocks the switching sleeve and the needle protection sleeve against renewed movement in the proximal direction.

A coupling sleeve 23 with two holding cams 23a is coupled to the spring coil 20b via coupling elements. Before discharge, the holding cams 23a engage in recesses of the axially fixed mechanical holder 13 and are prevented from moving outwards by an inner circumference of the locking sleeve 18, as a result of which the coupling sleeve 23 also cannot move axially. When the discharge is triggered, the locking sleeve 18 is moved away from the position of the recesses by a proximal movement of the needle protection sleeve 14 so that the holding cams 23a can detach radially and release the coupling sleeve 23. The latter moves in the proximal direction and releases the spring coil 20b for rotation, as described in detail in patent application PCTEP2021076923. At the proximal end of the autoinjector is the display with a rotating display element 25a with a contrast pattern in the form of parallel stripes, a circumferential transparent window 25b, and a proximal, non-transparent closure 25c.

FIGS. 3a and 3b show two isometric views of the display element 25a with an engagement element 25d for engagement with a non-rotating grid on the inner side of the window 25b or the closure 25d. The engagement element 25d is spring-mounted and creates a number of clicking sounds corresponding to the rasterization when the display element rotates during discharge. The display element has a shape that follows that of the window or is adapted to it, with a first, distal region and a second region adjoining it in the proximal direction. The first region is cylindrical, parallel to the longitudinal axis, while the second region has a steadily decreasing diameter.

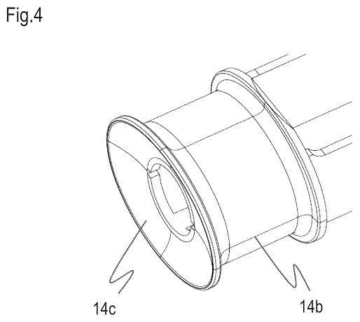

FIG. 4 shows the distal end of the needle protection sleeve with the sleeve-like portion 14b and the flange 14c. The shape of the flange is clearly visible with the concave curved contact surface, the outer, oval edge of which projects distally from the inner edge of the contact surface at the transition to the axis-parallel portion.

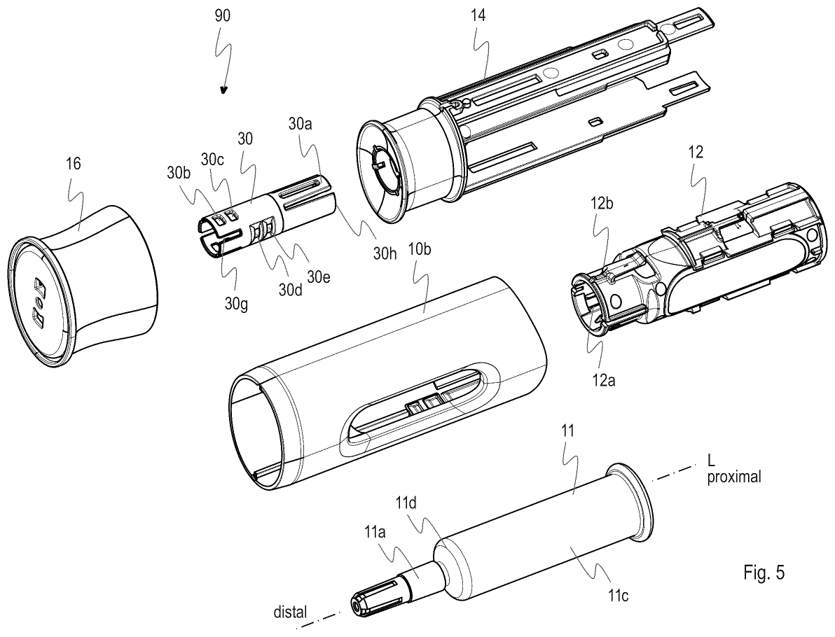

FIG. 5 is an exploded view of the components of a first embodiment of a product container receptacle 90, comprising a ready-to-use syringe 11 with a product container 11c which terminates distally by means of the shoulder 11d and with a needle protection cap 11a which encloses the injection needle, and comprising a device cap 16 which accommodates an insert 30. Two elastic snap arms with snap hooks 30a, two first and two second snap grooves 30b, 30c, two windows as snap connections 30d, 30e, two proximal end faces 30h, and four grooves 30g are formed on the sleeve-shaped insert 30. The ready-to-use syringe 11 can be accommodated in the syringe holder 12 through the proximal opening, and ten longitudinal ribs are attached on the inside of the guide sleeve 12b, which form a blocking surface 12a. Also shown are the needle protection sleeve 14 and a housing part 10b.

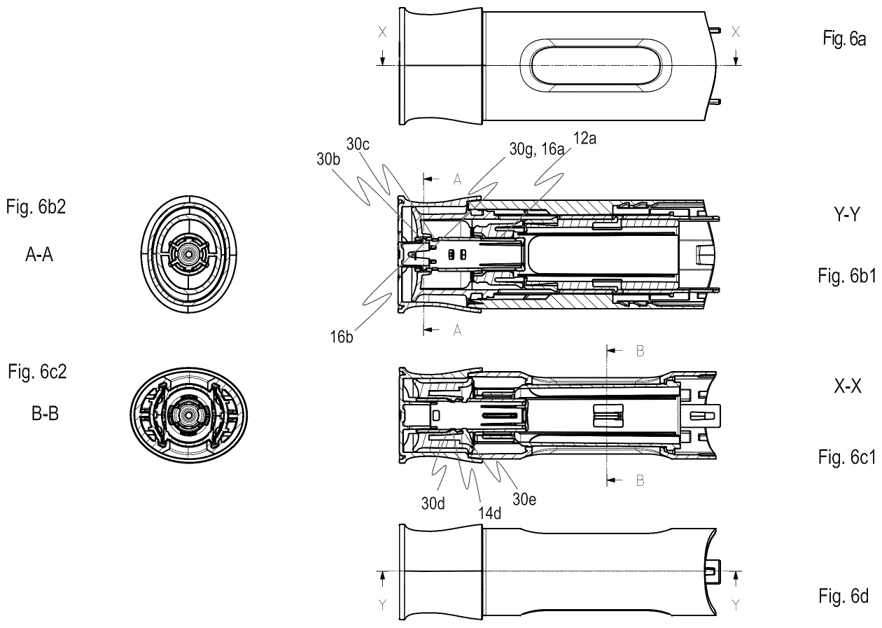

FIGS. 6a and 6d show the product container receptacle 90 of FIG. 5 in the state before final assembly of the autoinjector, while FIGS. 6b1 and 6c1 show two longitudinal sections, and FIGS. 6b2 and 6c2 show two radial cross-sections. The snap cams 16b inside the device cap 16 engage force-fittingly in the snap grooves 30b of the insert 30. Also visible are the four grooves 30g in which a rib 16a is slidably accommodated. The blocking surface 12a can be seen as a section through one of the ten ribs. The needle protection sleeve 14 engages with its two retaining catches 14d into the two openings 30d of the insert 30 and thus forms the first snap connection which releasably fixes the needle protection sleeve and the insert to one another.

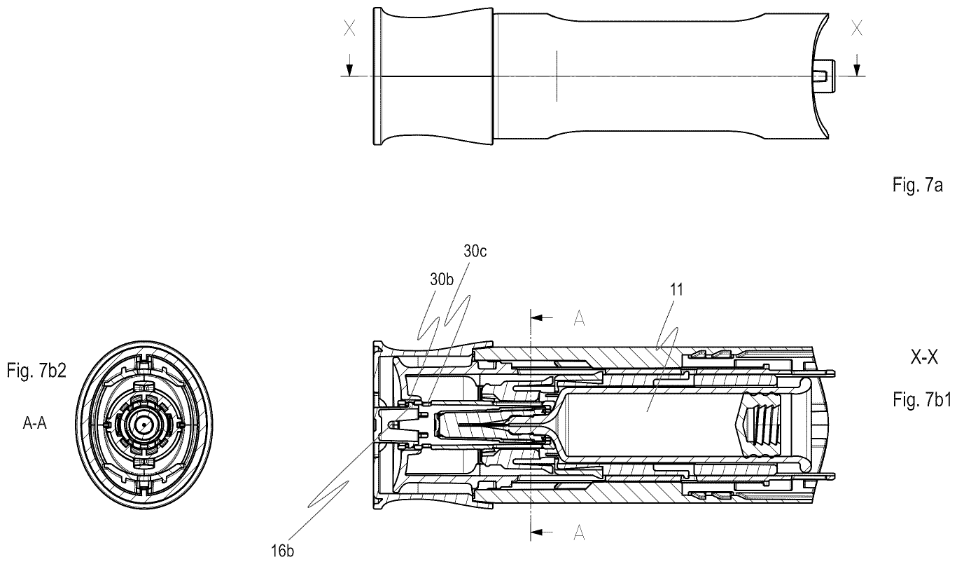

FIG. 7a shows the product container receptacle 90 of FIG. 5 in the state of joining the ready-to-use syringe 11 immediately before the blocking movement of the insert 30, while FIGS. 7b1 and 7b2 show longitudinal and radial cross-sections, respectively, of the product container receptacle 90 shown in FIG. 7a. This blocking movement from a first latching position to a second latching position of the insert 30 in the device cap 16 is caused by the joining movement of the ready-to-use syringe 11. In the first latching position, the snap cams 16b inside the device cap 16 engage force-fittingly in the snap grooves 30b of the insert 30. Before and at the beginning of the joining movement, the two elastic snap arms with snap hooks 30a do not overlap with the blocking surface 12a and can thus be deflected radially by the outer profile of the needle protection cap 11a until the snap hooks 30a snap in at the proximal end of the needle protection cap 11a.

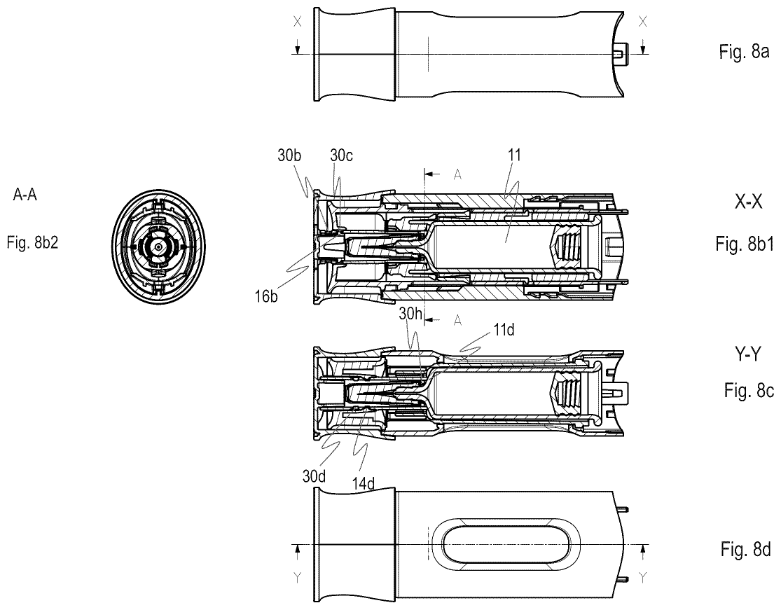

FIGS. 8a and 8d show the device of FIG. 5 in the state after the blocking movement of the insert 30, while FIGS. 8b1 and 8c show two longitudinal sections of FIGS. 8a and 8d, respectively, and FIG. 8b2 shows a radial cross-section of the device shown in FIG. 8a. In FIGS. 8a-8d, the ready-to-use syringe 11 causes the blocking movement during the joining process by displacing the insert 30 distally via its end faces 30h with the shoulder 11d, wherein the two elastic snap arms with snap hooks 30a on the insert 30 overlap with the blocking surface 12a and are thus blocked against radial deflection. After the blocking movement, in the second latching position, the snap cams 16b inside the device cap 16 now engage in a form-fitting manner in the snap grooves 30c of the insert 30, and the needle protection sleeve 14 now engages with its two retaining catches 14d in the two further openings 30e of the insert 30 and thus forms the second snap connection which releasably fixes the needle protection sleeve and the insert to one another.

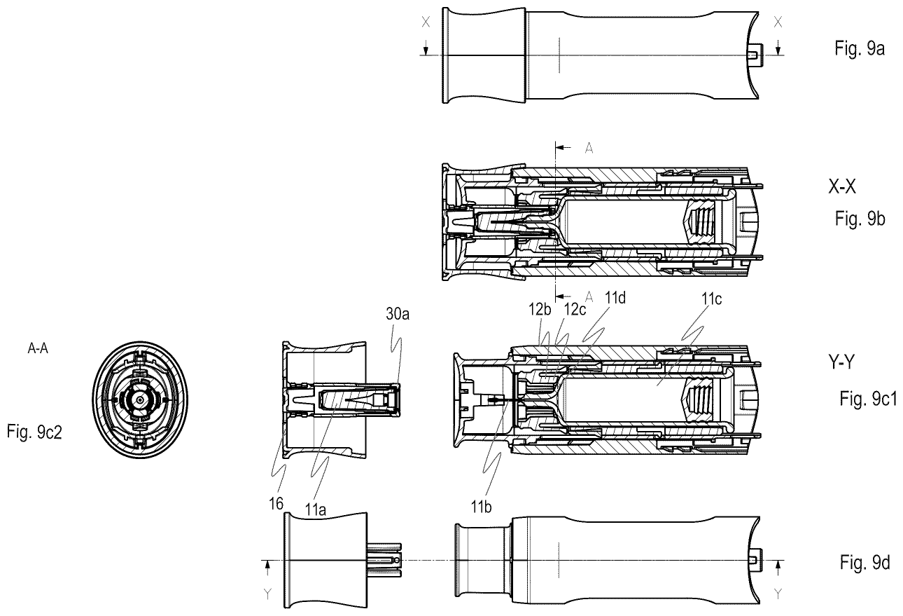

FIG. 9a shows the device of FIG. 5 in the state before the device cap 16 is pulled off, while FIG. 9d shows the device of FIG. 5 in the state after the device cap 16 is pulled off. FIGS. 9b and 9c1 show two longitudinal sections of FIGS. 9a and 9d, respectively, while FIG. 9c2 shows a radial cross-section of FIG. 9a. The pulling-off forces are transmitted from the ready-to-use syringe 11 via its shoulder 11d to the stop 12c of the guide sleeve 12b of the syringe holder 12, and, from there, to the housing. As shown by a comparison between FIGS. 9b and 9c1, when the device cap 16 is pulled off, the insert 30 remains fixedly connected to the device cap due to the form-fitting connection of the snap cams 16b inside the device cap 16 to the snap grooves 30c of the insert 30, and the two snap hooks 30a entrain the needle protection cap 11a and separate it from the ready-to-use syringe 11. This exposes the injection needle 11b.

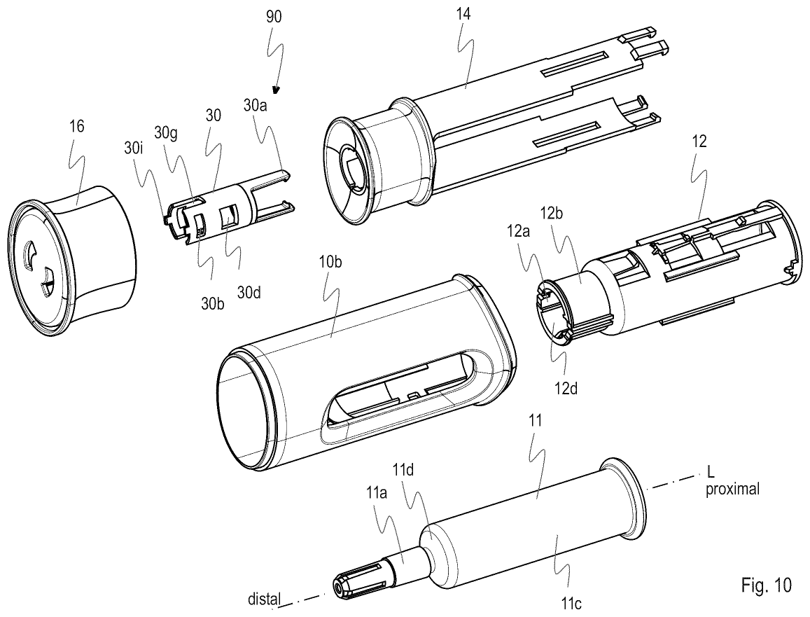

FIG. 10 is an exploded view of the components of a second embodiment of a product container receptacle 90, comprising a ready-to-use syringe 11 with product container 11c which terminates distally by means of the shoulder 11d, a needle protection cap 11a which encloses the injection needle, and a device cap 16 which accommodates the insert 30. Two elastic snap arms with snap hooks 30a, two snap grooves 30b, two windows or openings as snap connections 30d, two grooves 30g, and two assembly cams 30i are formed on the sleeve-shaped insert 30. The ready-to-use syringe 11 can be accommodated in the syringe holder 12 through a proximal opening, and a plurality of longitudinal ribs are attached to the inside of the guide sleeve 12b, which are distributed around the circumference and form the blocking surface 12a. The blocking surface 12a is interrupted by two recesses 12d in that the longitudinal ribs are missing at two opposing points on the inside of the guide sleeve 12b. Also shown are the needle protection sleeve 14 and a housing part 10b.

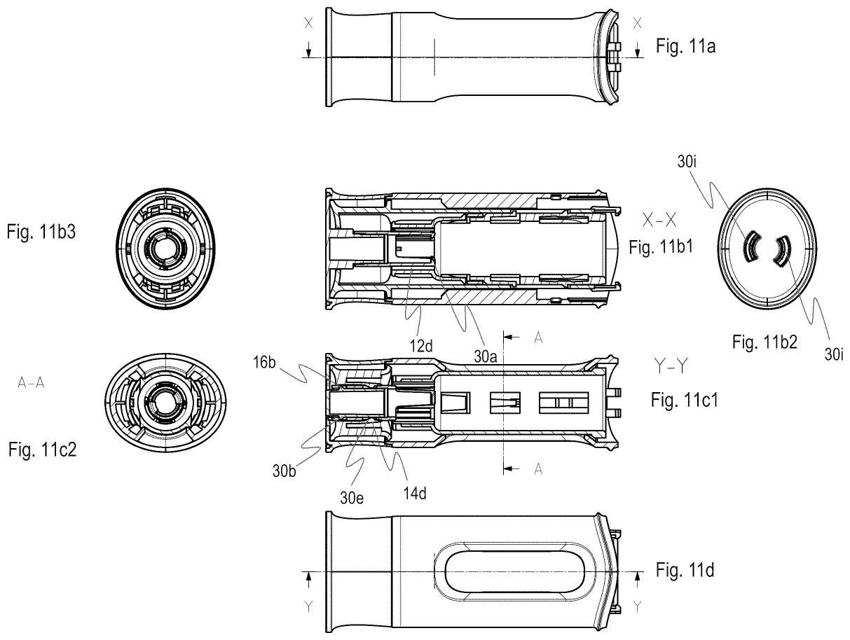

FIGS. 11a and 11d show of the product container receptacle 90 of FIG. 10 in the state before final assembly of the autoinjector, while FIGS. 11b1 and 11c1 show two longitudinal sections of FIGS. 11a and 11d, respectively, FIG. 11b2 shows a proximal view of the distal end of the device of FIG. 11a, and FIGS. 11b3 and 11c2 show radial cross-sections of the device of FIGS. 11a and 11d. The two snap cams 16b inside the device cap 16 engage form-fittingly in the snap grooves 30b of the insert 30. The recess 12d of the blocking surface 12a can be seen in section and releases the two elastic snap arms with snap hooks 30a for radial deflection. The needle protection sleeve 14 engages with its two retaining catches 14d into the two openings 30d of the insert 30 and thus forms the first snap connection which releasably fixes the needle protection sleeve and the insert to each other.

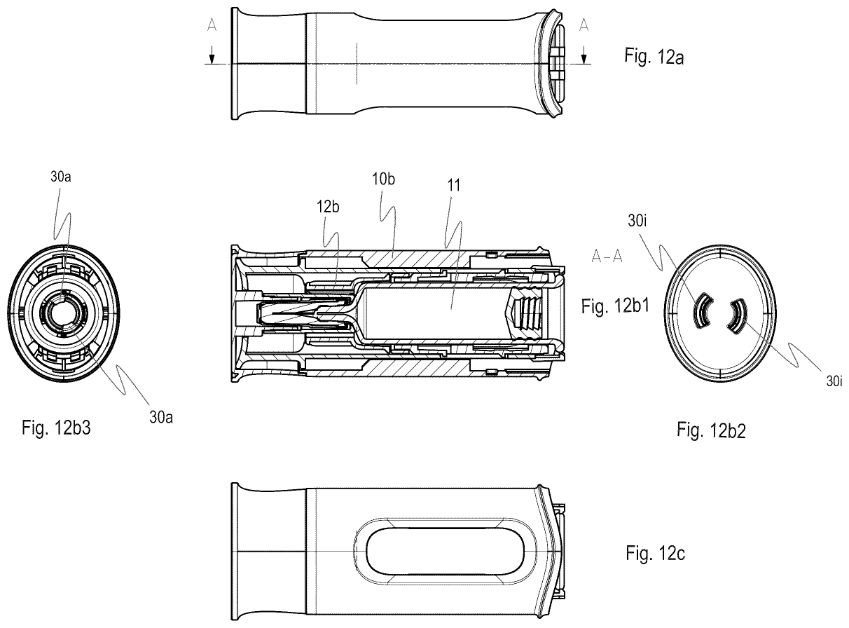

FIGS. 12a and 12c show of the product container receptacle 90 of FIG. 10 in the state of joining the ready-to-use syringe 11 immediately before the blocking movement of the insert 30, FIG. 12b1 shows a longitudinal section of FIG. 12a, FIG. 12b2 shows a proximal view of the distal end of the device of FIG. 12a, and FIG. 12b3 shows a cross-section of the device of FIG. 12a. The two snap cams 16b inside the device cap 16 engage axially and form-fittingly in the snap grooves 30b of the insert 30, wherein the insert 30 remains rotatable in the device cap. The assembly cams 30i are accessible to a turning tool via two openings in the device cap, and the two elastic snap arms with snap hooks 30a on the insert 30 can be deflected in the region of the recess 12d and thus radially by the outer profile of the needle protection cap 11a until the snap hooks 30a snap in at the proximal end of the needle protection cap 11a.

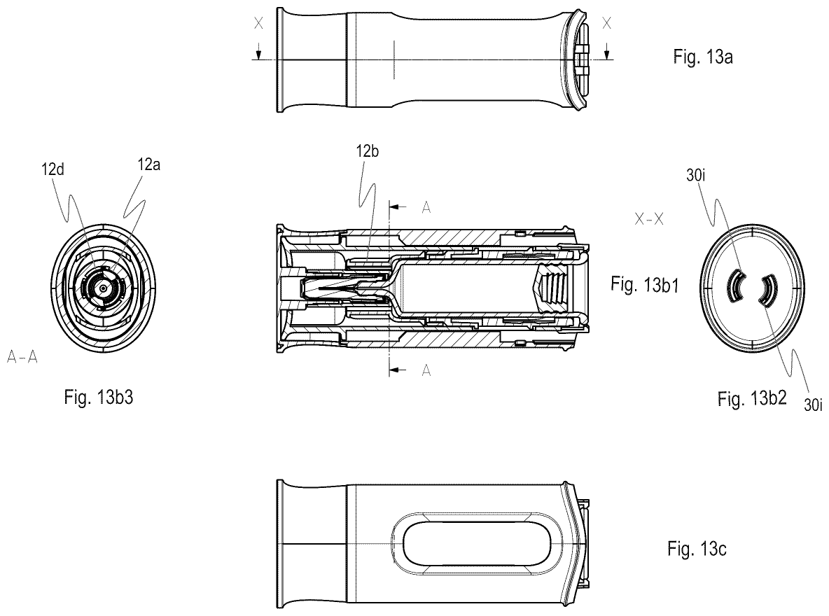

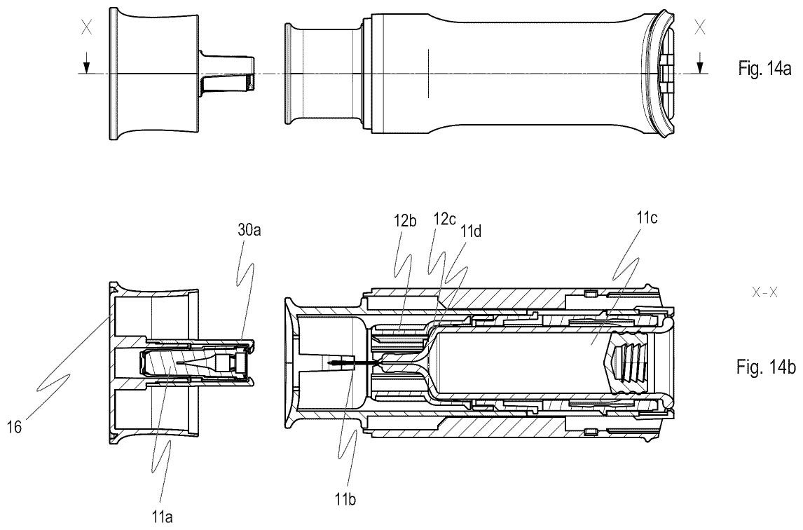

FIGS. 13a and 13c show of the product container receptacle 90 of FIG. 10 in the state after the blocking movement of the insert, wherein the insert 30 has been rotated relative to the device via the assembly cams 30i so that the two elastic snap arms with snap hooks 30a on the insert 30 overlap with the blocking surface 12a formed by the longitudinal ribs and are thus blocked against radial deflection, wherein this new rotational position of the insert 30 is secured by a snap connection between the device cap 16 and the groove 30g. FIG. 13b1 shows a longitudinal cross-section of the device of FIG. 13a, FIG. 13b2 shows a proximal view of the distal end of the device of FIG. 13a, and FIG. 13b3 shows a radial cross-section of the device of FIG. 13a.

FIG. 14a shows the device the product container receptacle 90 of FIG. 10 in the state after the needle protection cap is pulled off, and FIG. 14b shows a longitudinal cross-section of the device of FIG. 14a. The pulling-off forces are transmitted from the ready-to-use syringe 11 via its shoulder 11d to the stop 12c of the guide sleeve 12b of the syringe holder 12, and, from there, to the housing. When the device cap 16 is pulled off, the insert 30 remains fixedly connected to the device cap due to the form-fitting connection of the two snap cams 16b inside the device cap 16 to the snap grooves 30b of the insert 30, and the two snap hooks 30a entrain the needle protection cap 11a and separate it from the ready-to-use syringe 11. This exposes the injection needle 11b.

LIST OF REFERENCE SIGNS

| | | | |
|---|---|---|---|
| 10a, 10b | Housing part | 12c | Stop |
| 10c | Recess | 12d | Recess |
| 11 | Ready-to-use syringe | 90 | Product container receptacle |
| 11a | Needle protection cap | 16 | Device cap |
| 11b | Injection needle | 17 | Switching sleeve |
| 12 | Syringe holder | 18 | Locking sleeve |
| 13 | Mechanism holder | 18a | Locking link |
| 13a | Retaining spring portion | 20a | Spiral spring |
| 13b | Spring sleeve | 20b | Spring coil |
| 14 | Needle protection sleeve | 21 | Drive element |
| 14a | Arm | 22 | Propulsion element |
| 14b | Portion | 23 | Coupling sleeve |
| 14c | Flange | 3a | Holding cam |
| 15 | Needle protection spring | 25a | Display element |
| 30 | Insert | 25b | Window |
| 30a | Snap arm with snap hook | 25c | Closure |
| 30b | First snap groove | 25d | Engagement element |
| 30c | Second snap groove | 14d | Retaining catch |
| 30d | Opening, first snap connection | 16a | Rib |
| 30e | Opening, second snap connection | 16b | Snap cam |
| 30f | Guide groove | 16c | Latching cam |
| 30g | Groove | | |
| 30h | End face | | |
| 30i | Assembly cam | L | Longitudinal axis |
| 11c | Cylindrical product container | 1 | Autoinjector |
| 11d | Shoulder | | |
| 12a | Blocking surface, longitudinal ribs | | |
| 12b | Guide sleeve | | |

What is claimed is:

1. A product container receptacle for an autoinjector, comprising
a syringe holder configured to accommodate a pre-filled, ready-to-use syringe defining a longitudinal axis and having a cylindrical product container, which narrows at a distal end via a shoulder, and a needle protection cap;
a sleeve-shaped insert comprising a snap arm with a snap hook configured to be radially deflectable relative to the longitudinal axis; and
a device cap, wherein the insert is connectably accommodated in an interior of the device cap and is movable relative to the device cap from a first latching position to a second latching position in which the device cap connects to the insert, wherein in the second latching position, the insert is configured to be latching and form-fitting with respect to tensile forces along the longitudinal axis, and the device cap is configured to be separated from the product container receptacle by a pulling-off movement during which the insert and the needle protection cap are entrained,
wherein in the first latching position, the snap arm with the snap hook is radially deflectable, and in the second latching position, the snap arm with the snap hook is prevented from radial deflection by a blocking surface of the syringe holder formed on an inner wall of a distally, coaxially arranged guide sleeve.

2. The product container receptacle according to claim 1, wherein the snap arm with the snap hook is configured to be deflected radially outwards by the needle protection cap when the insert is in the first latching position.

3. The product container receptable according to claim 2, wherein the snap arm with snap hook is designed to axially entrain the needle protection cap in the distal direction in a form-fitting manner when the insert is in the second latching position.

4. The product container receptacle according to claim 1, wherein the snap arm with the snap hook is configured to axially entrain the needle protection cap in a distal direction in a form-fitting manner when the insert is in the second latching position.

5. The product container receptacle according to claim 1, wherein the syringe holder is configured such that the needle protection cap is axially movable in the guide sleeve.

6. The product container receptacle according to claim 5, wherein a proximal end stop of the guide sleeve is configured to limit movement of the shoulder of the product container in the distal direction in a form-fitting manner.

7. The product container receptacle according to claim 1, further comprising a needle protection sleeve comprising a retaining catch, wherein in the first latching position, the retaining catch is configured to establish a force-fitting connection between the insert and the needle protection sleeve by engaging in a first snap connection, and/or wherein in the second latching position, the retaining catch is configured to establish force-fitting connection between the insert and the needle protection sleeve by engaging in a second snap connection or by moving in the first snap connection, wherein the force-fitting connection between the needle protection sleeve and the insert is released by the pulling-off movement of the device cap.

8. The product container receptacle according to claim 1, further comprising a housing or a housing part, wherein the syringe holder is configured to be immovably accommodated in the housing or housing part, at least with respect to tension in the distal direction or pressure from a proximal direction, or is formed integrally therewith.

9. The product container receptacle according to claim 1, wherein the insert is movable axially relative to the device cap from a proximal end to the distal end from the first latching position to the second latching position, while a snap cam of the device cap undergoes a snapping movement from a first snap groove of the insert to a second snap groove of the insert.

10. The product container receptacle according to claim 9, wherein the insert is configured to be pushed axially in the distal direction by the shoulder via a proximal end face.

11. The product container receptable according to claim 10, wherein the blocking surface formed on the inner wall of the guide sleeve is limited to an axial portion.

12. The product container receptacle according to claim 9, wherein the blocking surface formed on the inner wall of the guide sleeve is limited to an axial portion.

13. The product container receptacle according to claim 1, wherein the insert is movable from the first latching position to the second latching position relative to the device cap in a rotational manner about the longitudinal axis and is axially fixedly guided by a snap cam in a snap groove and/or can be secured in a rotationally fixed manner by the groove in the second latching position.

14. The product container receptacle according to claim 13, wherein the insert is movable rotationally about the longitudinal axis via one or more distally provided assembly cams.

15. The product container receptable according to claim 14, wherein the blocking surface formed on the inner wall of the guide sleeve has one or more recesses in sectors.

16. The product container receptacle according to claim 13, wherein the blocking surface formed on the inner wall of the guide sleeve has one or more recesses in sectors.

17. The product container receptable according to claim 1, wherein the insert is movable from the first latching position to the second latching position relative to the device cap by means of a tool and/or a joining movement of the pre-filled ready-to-use syringe into the syringe holder.

18. The product container receptable according to claim 1, wherein the snap arm is prevented from radial deflection by a blocking surface of the syringe holder formed on the inner wall of a distally, coaxially arranged guide sleeve in the second latching position.

19. An autoinjector for administering a liquid product comprising:
the product container receptacle according to claim 1;
a propulsion member;
a drive element for moving the propulsion member along the longitudinal axis and for automatically discharging a liquid product contained in the product container of the pre-filled, ready-to-use syringe through an injection needle; and
a needle protection sleeve configured to protrude distally over a housing of the product container receptacle in a delivery state of the autoinjector,
wherein when the autoinjector is pressed against a point of injection, the autoinjector executes an actuating movement in the proximal direction, and
wherein when the autoinjector is removed from the point of injection, the autoinjector executes a needle protection movement in the distal direction in order to laterally surround the injection needle with a sleeve-shaped portion.

20. A method for assembling a product container receptacle of an autoinjector for administering a liquid product, comprising the steps of:
providing a syringe holder having a distally, coaxially arranged guide sleeve and a device cap which can be separated from the product container receptacle by a pulling-off movement, the product container receptacle configured to accommodate a pre-filled, ready-to-use syringe defining a longitudinal axis with a cylindrical product container, which narrows at a distal end via a shoulder, and a needle protection cap;
holding a sleeve-shaped insert in the device cap by latching the sleeve-shaped insert in a first axial latching position, the insert comprising at least one snap arm with a snap hook;
positioning the ready-to-use syringe coaxially with the syringe holder;
assembling the ready-to-use syringe in an axial joining movement in a distal direction through a proximal opening in the syringe holder, wherein the needle protection cap is guided through the guide sleeve and deflects the at least one snap arm with a snap hook radially outwards until the at least one snap arm with a snap hook snaps-in behind a proximal edge of the needle protection cap; and
securing the sleeve-shaped insert in the device cap by latching the sleeve-shaped insert in a second axial latching position, wherein movement of the insert from the first axial latching position to the second axial latching position is caused by or derived from the axial joining movement of the ready-to-use syringe, and wherein in the second latching position, the at least one snap arm is prevented from being deflected outwards by a blocking surface formed on an inner wall of the guide sleeve.
